(19) United States
(12) Patent Application Publication
(10) Pub. No.: US 2002/0022587 A1
(43) Pub. Date: Feb. 21, 2002

(54) Methods for effecting neuroprotection

(76) Inventors: Alastair V. Ferguson, Kingston (CA); Jaideep S. Bains, Calgary (CA)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/817,229

(22) Filed: Mar. 27, 2001

Related U.S. Application Data
(60) Provisional application No. 60/192,585, filed on Mar. 28, 2000.

(51) Int. Cl. A61K 31/00

(52) U.S. Cl. 514/1; 424/9.2

(57) ABSTRACT

The present invention relates to methods for preventing damage to excitable cells following ischemic by administering to a patient who is undergoing or who has undergone an ischemic event an effective amount of a compound which increases a transient potassium (K⁺) conductance in the excitable cells of the patient. The present invention also provides a method for screening for compounds which increase a transient K⁺ current in the excitable cells of a patient.

### CROSS-REFERENCE TO RELATED APPLICATIONS

This application claims benefit under 35 U.S.C. § 119(e) to provisional application 60/192,585, filed Mar. 28, 2000.

### BACKGROUND OF THE INVENTION

1. Field of the Invention

This invention relates to methods for preventing and reducing damage to excitable cells following ischemia.

2. Background Art

Cerebral ischemic events, commonly referred to as strokes, cause depolarization of the post-synaptic membrane of cerebral neurons. This initial depolarization causes the extracellular buildup of the excitotoxin glutamate (Nicholls and Atwell, Trends Pharmacol. Sci. 11:462-68 (1990)). The excess glutamate activates a variety of glutamate receptors, e.g. N-methyl-D-aspartate (NMDA) receptors, on the surface of these neurons, which results in prolonged depolarization of the post-synaptic membrane (Rothman and Olney, Trends Neurosci. 10(7):299-302 (1987)). Such prolonged depolarization results in impaired ion homeostasis and pathological membrane permeability changes which ultimately lead to neuronal death. Id.

Excitotoxins such as glutamate cause cell death in all brain areas. including the paraventricular nucleus (PVN) of the hypothalamus (Olney. J. Neuropathol. Exp. Neurol. 30(1):75-90 (1971)). The PVN is made up of parvocellular and magnocellular neurons. Of these two types ofneurons, only the parvocellular neurons die in response to glutamate excitotoxicity (Herman and Wiegand, Brain Res. 383:367-72 (1986); Hastings and Herbert, Neuroscience Lett. 69:1-6 (1986)). Previous studies have shown that, following activation of NMDA receptors of neurons in the PVN, parvocellular neurons demonstrate an increase in firing frequency, followed by a long-duration plateau depolarization (LDPD), while magnocellular neurons do not exhibit such a response (Bains and Ferguson, Eur. J. Neurosci. 10:1412-21 (1998)). Because parvocellular and magnocellular neurons both contain functional NMDA receptors (Hu and Bourque, J. Neuroendocrinol. 3:509-14 (1991)), the failure of magnocellular neurons to exhibit LDPD in response to activation of NMDA receptors may be due to differences in the intrinsic electrical properties of these neurons.

Magnocellular neurons are characterized by a rapidly activating-rapidly inactivating potassium (K⁺) current thought to be the A current (I_{A}) (Tasker and Dudek, J. Physiol. 434:271-93 (1991)). K⁺ currents, also referred to as K+conductances and K⁺ channels, are membrane-spanning proteins present in all neurons that allow the selective movement of K⁺ into or out of cells in response to changes in membrane potential, or in response to activation by cations including intracellular calcium (An et al., Nature 403:553-556 (2000)), and/or in response to a ligand. The primary role of K⁺ currents is maintenance of the resting membrane potential (Hodgkin et al., Arch. Sci. Physiol. 3:129-50(1949)). Another role concerns their contribution to depolarization of action potentials in excitable cells. Recent experiments have demonstrated that inhibition of I_{A} in magnocellular neurons by the compound 4-aminopyridine (4-AP) results in a change in membrane potential in these neurons similar to that observed in parvocellular neurons in response to NMDA agonist (Bains and Ferguson, supra). More important, however, is that these neurons die at a rate comparable to that of their parvocellular counterparts in response to glutamate excitoxicity (Id.).

Stroke is presently recognized as the third leading cause of adult disability and death in the United States and Europe. When a cerebral ischemic event occurs, neurons in the ischemic zone die quickly (Rothman and Olney, Ann. Neurol. 19:105-11 (1986)), a fact which makes these neurons an unlikely target of therapeutic manipulation. In contrast, neurons in the ischemic penumbra continue to die in the period immediately following ischemia despite the apparent restoration of acceptable vascular supply (Flaherty and Weisfeldt, Free Radic. Biol. Med. 5(5-6):409-19 (1988)). It is the death of these neurons which represents a major contribution to the pathology of ischemia victims (Bereczki et al., Eur. Arch. Psychiatry Neurol. Sci. 238(1):11-18 (1988)).

Despite the frequency of occurrence of ischemia and despite the serious nature of the outcome for the patient, treatments for these conditions have proven to be elusive. There are two basic approaches that have been undertaken to rescue degenerating cells in the penumbra. The first and most effective approach to date has been the identification of blood clot dissolvers that bring about rapid removal of the vascular blockage that restricts blood flow to the cells. Recombinant tissue plasminogen activator (TPA) has been approved by the Food and Drug Administration for use in dissolving clots that cause ischemia in thrombotic stroke. Nevertheless, adverse side effects are associated with the use of TPA. For example, a consequence of the breakdown of blood clots by TPA treatment is cerebral hemorrhaging that results from blood vessel damage caused by the ischemia. A second basic approach to treating degenerating cells deprived of oxygen is to protect the cells from damage that accumulates from the associated energy deficit. To this end, glutamate antagonists and Ca²⁺ channel antagonists have been most thoroughly investigated. None of these have proven to be substantially efficacious but they are still in early clinical development. No treatment other than TPA is currently approved for stroke.

Hypertension is one of the primary risk factors for ischemic stroke, although the exact mechanisms of this relationship remain unexplained. Hypertension is associated with increased circulating and central levels of angiotensin-II, a potent presser agent which exerts its action by a direct effect on arteriolar smooth muscle. Hypertension is currently treated by a variety of therapies, one of the more promising of which seeks to block either the production of angiotensin-II (Johnson et al., Clin. Sci Mol. Med. Suppl. 2:53-s56s (1975)) or its primary target, the AT₁ receptor (MacDonald et al., Clin. Exp. Pharmacol. Physiol. 2:89-91 (1975)). These therapies reduce the occurrence and severity of ischemic stroke independent of effects on blood pressure (See, e.g. Stier, et al., J. Hypertens. Supp. 11(3):S37-S42 (1993); Inada et al., Clin. Exp.

Hypertens. 19:1079-99 (1997); von Lutteroti et al., J. Hypertens. 10(9):949-957 (1992). Previous studies have demonstrated selective AT₁ receptor mediated inhibition of I_{A} in magnocellular neurons by angiotensin-II (Li and Ferguson, Neuroscience 71(1):133-45 (1996)). While it is obviously desirable to prevent ischemia from occurring in the first place, it is also important to ameliorate the damage following the occurrence of ischemia, particularly in light of the major role played by penumbric neuronal death in the pathology of victims of ischemia.

### BRIEF SUMMARY OF THE INVENTION

The present invention is based at least in part on the discovery that the magnocellular neurons in hypertensive subjects with increased central angiotensin-II lose their resistance to glutamate excitotoxicity as a consequence of endogenous angiotensin-II inhibiting I_{A}. The present invention is further based on the discovery that damage to excitable cells following ischemia is prevented by agents which interfere with AT₁ receptor-mediated inhibition of cellular K⁻ currents, particularly transient K⁺ currents.

The present invention provides a method of preventing damage to the excitable cells of a patient which comprises administering to said patient during or after said patient undergoes or has undergone an ischemic event, an effective amount of a compound which increases a transient K⁺ current in the excitable cells of said patient.

The present invention also provides a method of preventing damage to the excitable cells of a patient which comprises administering to said patient during or after said patient undergoes or has undergone an ischemic event, an effective amount of an angiotensin-II receptor antagonist which increases a transient K⁻ current in the excitable cells of said patient.

The present invention also provides an in vivo method for screening for compounds that increase a transient K⁺ current in the excitable cells of a patient. comprising the steps of: (i) inducing ischemia in a subject; (ii) assessing a transient K⁺ current in the subject; (iii) administering to the subject an effective amount of a test compound; and (iv) assessing the transient K⁺ current in the subject, wherein an increase in the transient K⁺ current indicates that the test compound increases a transient K⁺ current in the excitable cells of a patient.

The present invention also provides an in vitro method for screening for compounds that increase a transient K⁺ current in the excitable cells of a patient. comprising the steps of: (i) inducing an oxygen-deprived state mimicking ischemia in an isolated cell; (ii) assessing a transient K⁺ current in the cell; (iii) administering to the cell an effective amount of a test compound; and (iv) assessing the transient K⁺ current in the cell, wherein an increase in the transient K⁺ current indicates that the test compound increases a transient K⁺ current in the excitable cells of a patient.

In a specific embodiment of this invention, the excitable cells-are the neurons of the brain.

In another specific embodiment of this invention, the excitable cells are the magnocellular neurons of the paraventricular nucleus of the hypothalamus.

In another specific embodiment of this invention, the transient K⁺ current is I_{A}.

In another specific embodiment of this invention, the transient K⁺ current is I_{D}.

In another specific embodiment ofthis invention, the transient K⁺ current is I_{A} and I_{D}.

In another specific embodiment of this invention, the transient K⁺ current iS I_{TO}.

In a preferred embodiment of this invention, the compound crosses the blood-brain barrier.

In another specific embodiment of this invention, the compound is a vasopressin receptor antagonist.

In another specific embodiment ofthis invention, the vasopressin receptor antagonist crosses the blood-brain barrier.

In another specific embodiment of this invention, the compound is an angiotensin converting enzyme (ACE) inhibitor.

In another specific embodiment of this invention, the angiotensin converting enzyme (ACE) inhibitor crosses the blood-brain barrier.

In another specific embodiment of this invention, the angiotensin-II receptor antagonist crosses the blood-brain barrier.

In another specific embodiment of this invention, the angiotensin-II receptor antagonist that crosses the blood-brain barrier is losartan.

In another specific embodiment of this invention, the angiotensin-II receptor antagonist is saralasin.

Further features, objects, and advantages of the present invention will become more fully apparent to one of ordinary skill in the art from a detailed consideration of the following description of the invention when taken together with the accompanying drawings.

### BRIEF DESCRIPTION OF THE DRAWINGS/FIGURES

FIG. 1. FIG. 1 depicts whole-cell recordings which illustrate the cellular response to application of 1 μM NMDA agonist in coronal hypothalamic slices. Typical responses from magnocellular (top) and parvocellular (bottom) neurons are shown.

FIG. 2 depicts histological coronal sections through rat PVN (scale bar 75 μm) following microinjection of NMDA (left) and NMDA in the presence of 4-AP. A statistically significant reduction in magnocellular neuron numbers in PVN treated with 4-AP is seen as summarized in the bar graph on the right (N values indicated for each group; *=p<0.05).

FIG. 3. FIG. 3 depicts histological coronal sections through Sprague-Dawley rat PVN (scale bar 75 μm) following microinjection of NMDA (left) and NMDA in the presence of angiotensin-II (right). NMDA results in the loss of parvocellular neurons only, while in the presence of angiotensin-II, cell loss is observed also in magnocellular cell groups as summarized in the bar graph on the right (N values indicated for each group; **=p<0.01).

FIG. 4. FIG. 4 depicts histological coronal sections through SHR PVN (scale bar 75 μm) following microinjection of NMDA (left) and NMDA in the presence of the angiotensin-II receptor antagonist saralasin (right). Microinjection of NMDA induces cell death in both magnocellular and parvocellular neurons. Application of NMDA in the presence of saralasin, however, prevents cell death in magnocellular neurons. Saralasin by itself has no effect on cell viability as summarized in the bar graph on the right (N values indicated for each group; **=p<0.01).

FIG. 5. FIG. 5 depicts voltage clamp recordings of isolated PVN neurons, that demonstrate the presence of I_{A} and I_{D} currents in PVN neurons.

FIG. 5a(iii) represents the I_{A} component derived arithmetically by subtracting the slower and inactive activation components (a(ii)) from the rapid activation/inactivation components (a(i)). The I_{D} voltage component is obtained by subtracting recordings of currents from cells blocked with 4-AP from non-blocked cells (a(iv)). FIG. 5a(v) shows normalized traces at the same potential (10 mV) to distinguish between the 3 types of K⁺ currents.

FIG. 5b shows that voltage ramps that activate outwardly-rectifying whole-cell currents are inhibited to an equal degree with 100 μM of 4-AP and 1 μM of A-DTX.

### DETAILED DESCRIPTION OF THE INVENTION

1. Definitions

In order to provide a clearer and more consistent understanding of the invention, the following definitions are provided.

As used herein, “preventing” is intended to refer to eliminating, avoiding. ameliorating, diminishing, treating, and reducing ischemia-induced cellular damage and/or symptoms associated with dysfunction of cellular membrane polarization and conductance. The term “preventing” as used herein also covers any treatment of ischemia-induced cellular damage in a mammal, especially a human, and includes: (i) preventing ischemia-induced cellular damage from occurring in a subject which may be predisposed to the disease but which may or may not have yet been diagnosed as having it; (ii) inhibiting ischemia-induced cellular damage, i.e. arresting its development; or (iii) relieving ischemia-induced cellular damage, i.e. causing regression of the disease. Cellular damage is “prevented” if there is a reduction in the amount of cell death that would have been expected to have occurred but for the administration of a compound of the invention. The term “preventing” as used herein is also meant to refer to the process of effecting neuroprotection.

As used herein, “damage” is intended to refer to ischemia-induced cellular injury, impairment, deterioration, and death.

As used herein, “excitable cells” is intended to refer to mammalian cells specialized for the transmission of electrical signals, including neurons, such as interneurons, sensory neurons, and motor neurons, and cardiac myocytes. This term is also intended to encompass the magnocellular and parvocellular neurons of the paraventricular nucleus of the hypothalamus.

As used herein, “patient” and “subject” are intended to refer to a mammal, especially a human, whose excitable cells are susceptible to damage as the result of suffering an ischemic event.

As used herein, “administering” is intended to refer to orallv. intravenously, intramuscularly, intraperitoneally, intradermally, subcutaneously. sublingually, buccally, rectally or in any other acceptable manner delivering to a patient who is suffering from or who has recently suffered an ischemic event. a compound to prevent cellular damage in the patient following an ischemic event. This term is also meant to encompass intramucosal delivery, including by aerosol.

As used herein, “during or after said patient undergoes or has undergone an ischemic event” is intended to refer the period of time between the onset of an ischemic event, characterized by membrane depolarization in the excitable cells of the patient who is suffering from the ischemic event, and the cessation of an ischemic event, characterized by membrane repolarization in the excitable cells of a patient who has recently suffered an ischemic event, as well as the seconds. minutes, hours, and days following the cessation of an ischemic event in a patient who has suffered an ischemic event.

As used herein, “ischemia” is intended to refer to an acute condition associated with an inadequate flow of oxygenated blood to a part of the body, caused by the constriction or blockage of the blood vessels supplying it. Global ischemia occurs when blood flow to an entire organ ceases for a period of time. such as may result from cardiac arrest. Focal ischemia occurs when a portion of an organ is deprived of its normal blood supply, such as may result from: (i) the blockage of a vessel by an embolus (blood clot); (ii) the blockage of a vessel due to atherosclerosis; (iii) the breakage of a blood vessel (a bleeding stroke); (iv) the blockage of a blood vessel due to vasoconstriction such as occurs during vasospasms and possibly, during transient ischemic attacks and following subarachnoid hemorrhage. Conditions in which ischemia occurs further include:

(i) during myocardial infarction (when the heart stops, the flow of blood to organs is reduced and ischemia results); (ii) trauma; and (iii) during cardiac and neurosurgery (blood flow needs to be reduced or stopped to achieve the aims of surgery). Even if transient, both global and focal ischemia can produce widespread cellular damage. In the case of cerebral ischemia, although nerve tissue damage occurs over hours or even days following the onset of ischemia, some permanent nerve tissue damage may develop in the initial minutes following cessation of blood flow to the brain. Much ofthis damage is attributed to glutamate toxicity and secondary consequences of reperfusion of the tissue, such as the release of vasoactive products by damaged endothelium, and the release of cytologic products, such as free radicals and leukotrienes, by the damaged tissue.

When an ischemic event occurs, there is a gradation of injury that arises from the ischemic site. The cells at the site of blood flow restriction undergo necrosis and form the core of a lesion. A penumbra is formed around the core where the injury is not as immediately fatal but slowly progresses to cell death. This progression to cell death may be reversed upon reestablishment of blood flow within a short time of the ischemic event. As the blood flow is depleted, excitable cells fall electrically silent, their ionic gradients decay, the cells depolarize, and then die. In the case of cerebral ischemia, endothelial cells of the brain capillaries undergo swelling and the luminal diameter of the capillaries decrease. Associated with these events, the blood-brain barrier appears to be disrupted, and an inflammatory response follows which further interrupts blood flow and the access of cells to oxygen. The pathophysiology and treatment of focal cerebral ischemia has been reviewed by Seisjo (J. Neurosurgery 77:169-184 and 337-354 (1992)). The term “ischemia” is also intended to include the terms “cerebral ischemia,” “stroke,” “ischemic event,” and “cerebral ischemic event.”

As used herein, “an effective amount” is intended to refer to the total amount of the active compound of the method that is sufficient to show a meaningful patient benefit. This term is also intended to refer to an amount that returns to normal, either partially or completely, physiological or biochemical parameters associated with ischemia-induced cellular damage. A non-limiting example of an effective dose range for a therapeutic composition of the invention is 0.01-500 mg/kg of body weight per day, more preferably 0.01-50 mg/kg of body weight per day, and still more preferably 0.05-50 mg/kg of body weight per day. In an aqueous composition preferred concentrations for the active compound are 10 μM-500 mM, more preferably 10 μM-100 mM, still more preferably 10 μM-50 mM, still more preferably 50 μM-50 mM, and still more preferably 100 μM-50 mM.

As used herein, “compound” is intended to refer to an agent such as an organic drug, preferably a low molecular weight organic drug, or a higher molecular weight polypeptide or polynucleotide, as long as it causes an increase in a transient K⁺ current, directly or indirectly, in the excitable cells of a mammal. Although in no way meant to be limiting, specific examples of such agents are any of the angiotensin-II receptor antagonists, both peptidergic and nonpeptidergic, and any of the vasopressin receptor antagonists, such as EP-343 and OP-21268, or ACE inhibitors.

As used herein, “increases a transient K⁺ current” is intended to refer to any enhancement in the activity of a transient K current in the excitable cells of a mammal, especially a human. This phrase is also meant to include the opening of a transient K⁺ current. More specifically, this phrase refers to an increased flow of K⁺ ions from inside an electrically excitable cell to outside the cell via a membrane of the cell which has at least one transient K⁺ current. Transient K+current enhancing activity may be observed by measuring an increase in the flow of K⁺ ions from inside a cell to outside the cell via a transient K⁺ current in the cell membrane. The phrase “increases a transient K⁺ current” is also meant to encompass derepression of an inhibited transient K⁺ current.

As used herein, “transient K⁺ current” is intended to refer to a membranespanning protein present in the excitable cells of a mammal that regulates the movement of K⁺ ions into and out of such cells in response to changes in membrane potential, or in response to activation by cations, ligand, and/or signal transduction pathway factors. This term is also intended to include the terms “transient K⁺ channel” and “transient K conductance.” Several K channel types are opened in response to depolarization of the membrane during an action potential, and the currents carried by these different channels sum to cause depolarization of the membrane to the resting potential. The opening of voltage-dependent K⁺ channels is also the mechanism by which depolarization of the cell membrane occurs during the very short action potential characteristic of central neurons. Transient outward K⁺ currents, such as I_{A}, I_{D}, and I_{TO} play a role in this process.

K⁺ channels are structurally and functionally diverse families of K⁺ selective channel proteins which are ubiquitous in cells, indicating their central importance in regulating a number of key cell functions (Rudy, Neuroscience 25:729-749(1988)). K⁺ channels are important regulators of numerous biological processes, including secretory processes, muscle contraction, and post-ischemia cardioprotection. Electrophysiological studies have disclosed the existence of K⁺ channels in nearly all cell types (Gopalakrishnan et al., Drug Dev. Res. 28:95-127 (1993)). Such channels are present in various forms that are generally distinguishable by their respective structural, biophysical, electrochemical, and pharmacological characteristics (Id.). It is generally well known that the opening of K⁺ channels in a electrically excitable cell having such channels results in an increased flow of K⁺ ions from inside the cell to outside the cell. This flow of K⁺ ions causes a measurable change in the resting membrane potential of the cell and leads to membrane hyperpolarization and relaxation of the cell. Activation of K⁺ channels stabilizes cell membrane potential and generally reduces cell excitability. In addition to acting as an endogenous membrane voltage clamp, K⁺ channels can respond to important cellular events such as changes in the intracellular concentration of ATP or the intracellular concentration of Ca²⁻. The central role of K⁺ channels in regulating numerous cell functions makes them particularly important targets for therapeutic development (Cook, Potassium channels: Structure, classification, function and therapeutic potential; Ellis Norwood, Chinchester (1990)).

K⁺ channels have been implicated in a large number of diseases, including cardiovascular disease, asthma, hypertension, Parkinson's disease, Alzheimer's disease, diabetes, epilepsy, high blood pressure, and feeding and appetite disorders (See, e.g. Gopalakrishnan et al., supra; Ben-Ari et al., Neuroscience 37:55-60 (1990); Gandolfo et al., Eur. J. Pharmacol. 159(3):329-30 (1989); Ashford et al., Nature 370:456-59 (1994)). It is generally believed that inhibition of these K⁺ channels or disruption in the processes that activate such K⁺ channels may play a significant role in the pathogenesis of such diseases and illnesses. As a result, compounds that are of assistance in opening K⁺ channels and, consequently, in modulating electrophysiological functioning of the cells may have significant therapeutic and prophylactic potential for treating or alleviating such conditions.

K⁺ channel openers may also benefit brain tissues through their vasodilating properties. Some neurodegenerative diseases are characterized, at least in part, by a lack of oxygen and nutrients in neuronal tissue. It is known that a progressive lack of oxygen and nutrients in brain and neuronal tissues promotes the progression of neurodegenerative disease. By improving the delivery of oxygen and nutrients to neuronal tissue, neurodegenerative diseases may be slowed and stabilized. Vasodilation generally increases circulation and blood flow and improves oxygen and nutrient delivery to body tissues. With their vasodilating effects, K⁺ channel openers may assist in retarding and stabilizing neurodegenerative diseases, by increasing the flow of oxygen and nutrients to brain tissues in need thereof.

As used herein, “I_{A}” is intended to refer to a 4-AP-sensitive, rapidly activating-rapidly inactivating K⁺ current present in the neurons of a mammal. The term “I_{A}” is also intended to include the term “A current.” I_{A} is activated in the subthreshold voltage range more positive to −65 mV, and shows steep voltage dependence of inactivation, reaching maximal inactivation at approximately −40 mV (Hille, Ionic Channels of Excitable Membranes, Sinauer Associates, Inc., Massachusetts (1992)). This current is almost ubiquitous in excitable cells (Rogawski, Trends Neurosci. 8:214-19 (1985). I_{A} can be abolished by low doses of 4-AP, and is also sensitive to tetraethylammonium (TEA) to a lesser degree (Li and Ferguson, supra; Nagatomo et al., J. Physiol. (London) 485:87-96 (1995)). Functionally, the initial depolarizing phase of an action potential moves the membrane to the I_{A} activation range. The rapidly activating outward current opposes the depolarizing tendency, thus serving to dampen the initial firing response. In addition, the duration of activation of this current also means that it contributes significantly to the depolarization which occurs following an action potential as reflected by the distinct afterhyperpolarizations (AHP), which are also abolished by 4-AP (Bains and Ferguson, supra). Clearly, modulation of the voltage dependent gating of I_{A} can have profound effects on neuronal firing patterns.

As used herein, “I_{D}” is intended to refer to a rapidly activating-slowly inactivating K⁺ current present in the neurons of a mammal. The term “I_{D}” is also intended to include the terms “D current” and “delay current.” I_{D} has been described in detail by Storm (Nature 336:379-381 (1988)).

Active K⁺ conductances in magnocellular and parvocellular neurons can be characterized by step voltage clamp protocols in order to measure currentvoltage relations, and activation and inactivation properties (Li and Ferguson, supra; Fedida and Giles, J. Physiol. (London) 442:192-209 (1991); Bouchard and Fedida, J. Pharmacol. & Exp. Therap. 275:864-76 (1995)). K⁺ channel blockers such as TEA, 4-AP, or apamin/charybdotoxin are perfused into the bath to enable characterization of the pharmacological sensitivity of the Kv subunits expressed in magnocellular and parvocellular neurons of the PVN.

As used herein, “I_{TO}” is intended to refer to a rapidly activating-rapidly inactivating K⁺ current present in the cardiac myocytes of a mammal. I_{TO} contributes most significantly to initial depolarization of the cardiac action potential. I_{TO} has been described in detail by Escande et al. (Am. J. Physiol. 252:H142 (1987)).

As used herein, “angiotensin-II receptor antagonist” is intended to refer to a compound that competitively inhibits or interferes with the binding of angiotensin-II to an angiotensin-II receptor.

Angiotensin-II receptor antagonists are well known and include peptide and nonpeptide compounds. Most angiotensin-II receptor antagonists are slightly modified congeners in which agonist activity is attenuated by replacement of phenylalanine in position 8 of angiotensin-II with some other amino acid; stability can be enhanced by other replacements that slow degeneration in vivo.

The term “angiotensin-II receptor antagonist” is also intended to encompass the angiotensin-II receptor antagonists as recited in European patent applications: EP 475,206, EP 497,150, EP 539,096, EP 539,713, EP 535,463, EP 535,465, EP 542,059, EP 497,121, EP 535,420, EP 407,342, EP 415,886, EP 424,317, EP 435,827, EP 433,983, EP 475,898, EP 490,820, EP 528,762, EP 324,377, EP 323,841, EP 420,237, EP 500,297, EP 426,021, EP 480,204, EP 429,257, EP 430,709, EP 434,249, EP 446,062, EP 505,954, EP 524,217, EP 514,197, EP 514,198, EP 514,193, EP 514,192, EP 450,566, EP 468,372, EP 485,929, EP 503,162, EP 533,058, EP 467,207, EP 399,731, EP 399,732, EP 412,848, EP 453,210, EP 456,442, EP 470,794, EP 470,795, EP 495,626, EP 495,627, EP 499,414, EP 499,416, EP 499,415, EP 511,791, EP 516,392, EP 520,723, EP 520,724, EP 539,066, EP 438,869, EP 505,893, EP 530,702, EP 400,835, EP 400,974, EP 401,030, EP 407,102, EP 411,766, EP 409,332, EP 412,594, EP 419,048, EP 480,659, EP 481,614, EP 490,587, EP 467,715, EP 479,479, EP 502,725, EP 503,838, EP 505,098, EP 505,111, EP 513,979, EP 507,594, EP 510,812, EP 511,767, EP 512,675, EP 512,676, EP 512,870, EP 517,357, EP 537,937, EP 534,706, EP 527,534, EP 540,356, EP 461,040, EP 540,039, EP 465,368, EP 498,723, EP 498,722, EP 498,721, EP 515,265, EP 503,785, EP 501,892, EP 519,831, EP 532,410, EP 498,361, EP 432,737, EP 504,888, EP 508,393, EP 508,445, EP 403,159, EP 403,158, EP 425,211, EP 427,463, EP 437,103, EP 481,448, EP 488,532, EP 501,269, EP 500,409, EP 540,400, EP 005,528, EP 028,834, EP 028,833, EP 411,507, EP 425,921, EP 430,300, EP 434,038, EP 442,473, EP 443,568, EP 445,811, EP 459,136, EP 483,683, EP 518,033, EP 520,423, EP 531,876, EP 531,874, EP 392,317, EP 468,470, EP 470,543, EP 502,314, EP 529,253, EP 543,263, EP 540,209, EP 449,699, EP 465,323, EP 521,768, and EP 415,594, which are incorporated by reference into the instant application.

The term “angiotensin-II receptor antagonist” is also intended to encompass include the angiotensin-II receptor antagonists as recited in PCT patent applications: WO 92/14468, WO 93/08171, WO 93/08169, WO 91/00277. WO 91/00281, WO 91/14367, WO 92/00067, WO 92/00977, WO 92/20342. WO 93/04045, WO 93/04046, WO 91/15206, WO 92/14714, WO 92/09600, WO 92/16552, WO 93/05025, WO 93/03018, WO 91/07404, WO 92/02508, WO 92/13853, WO 91/19697, WO 91/11909, WO 91/12001, WO 91/11999, WO 91/15209, WO 91/15479, WO 92/20687, WO 92/20662, WO 92/20661, WO 93/01177, WO 91/17771, WO 91/14679, WO 91/13063, WO 92/13564, WO 91/17148, WO 91/18888, WO 91/19715, WO 92/02257, WO 92/04335, WO 92/05161, WO 92/07852, WO 92/15577, WO 93/03033, WO 91/16313, WO 92/00068, WO 92/02510, WO 92/09278, WO 9210179, WO 92/10180, WO 92/10186, WO 92/10181, WO 92/10097, WO 92/10183, WO 92/10182, WO 92/10187, WO 92/10184, WO 92/10188, WO 92/10180, WO 92/10185, WO 92/20651, WO 93/03722, WO 93/06828, WO 93/03040, WO 92/19211, WO 92/22533, WO 92/06081, WO 92/05784, WO 93/00341, WO 92/04343, WO 92/04059, and WO 92/05044, which are incorporated by reference into the instant application.

The term “angiotensin-II receptor antagonist” is also intended to encompass the angiotensin-II receptor antagonists as recited in U.S. patents: U.S. Pat. No. 5,104,877, U.S. Pat. No. 5,187,168, U.S. Pat. No. 5,149,699, U.S. Pat. No. 5,185,340, U.S. Pat. No. 4,880,804, U.S. Pat. No. 5,138,069, U.S. Pat. No. 4,916,129, U.S. Pat. No. 5,153,197, U.S. Pat. No. 5,173,494, U.S. Pat. No. 5,137,906, U.S. Pat. No. 5,155,126, U.S. Pat. No. 5,140,037, U.S. Pat. No. 5,137,902, U.S. Pat. No. 5,157,026, U.S. Pat. No. 5,053,329, U.S. Pat. No. 5,132,216, U.S. Pat. No. 5,057,522, U.S. Pat. No. 5,066,586, U.S. Pat. No. 5,089,626, U.S. Pat. No. 5,049,565, U.S. Pat. No. 5,087,702, U.S. Pat. No. 5,124,335, U.S. Pat. No. 5,102,880, U.S. Pat. No. 5,128,327, U.S. Pat. No. 5,151,435, U.S. Pat. No. 5,202,322, U.S. Pat. No. 5,187,159, U.S. Pat. No. 5,198,438, U.S. Pat. No. 5,182,288, U.S. Pat. No. 5,036,049, U.S. Pat. No. 5,140,036, U.S. Pat. No. 5,087,634, U.S. Pat. No. 5,196,537, U.S. Pat. No. 5,153,347, U.S. Pat. No. 5,191,086, U.S. Pat. No. 5,190,942, U.S. Pat. No. 5,177,097, U.S. Pat. No. 5,212,177, U.S. Pat. No. 5,208,234, U.S. Pat. No. 5,208,235, U.S. Pat. No. 5,212,195, U.S. Pat. No. 5,130,439, U.S. Pat. No. 5,045,540, and U.S. Pat. No. 5,210,204, which are incorporated by reference into the instant application.

The renin-angiotensin system (RAS) plays a central role in the regulation of normal blood pressure and seems to be critically involved in hypertension development and maintenance as well as congestive heart failure. Angiotensin-II is an octapeptide hormone produced mainly in the blood during the cleavage of angiotensin-I by angiotensin converting enzyme (ACE) localized on the endothelium of blood vessels of lung, kidney, and many other organs. It is the end product of the RAS and is a powerful arterial vasoconstrictor that exerts its action by interacting with specific receptors present on cell membranes. One of the possible modes of controlling the RAS is angiotensin-II receptor antagonism.

As mentioned, there exist both peptide and non-peptide angiotensin-II receptor antagonists. Several peptide analogs of angiotensin-II are known to inhibit the effect of this hormone by competitively blocking the receptors (See, e.g. Antonaccio, Clin. Exp. Hypertens. A4:27-46 (1982); Streeten and Anderson. Handbook of Hypertension, Clinical Pharmacology of Antihypertensive Drugs. ed. A. E. Doyle, Vol. 5, pp. 246-271, Elsevier Science Publisher, Amsterdam. The Netherlands (1984)). One such analog is the compound saralasin. Pals et al. (Circulation Res. 29:673 (1971)) describe the introduction of a sarcosine residue in position 1 and alanine in position 8 of the endogenous vasoconstrictor hormone angiotensin-II to yield an octapeptide that blocks the effects of angiotensin-II on the blood pressure of pithed rats. This analog, Sar¹Ala⁸-angiotensin-II, initially called “P-113” and subsequently “saralasin,” was found to be one of the most potent competitive antagonists of the actions of angiotensin-II. Another example of a peptide angiotensin-II receptor antagonist is CGP 42112 A (Nasjletti and Mason, Proc. Soc. Exp. Biol. and Med. 142:307-310 (1973)).

Non-peptide angiotensin-II receptor antagonists include: losartan [2-butyl-4-chloro-1 -[p-(o-1H-tetrazol-5-ylphenyl)-benzyl]imidazole-5-methanol monopotassium salt]; valsartan [N-(1-oxopentyl)-N-[[2′-(1H-tetrazol-5-yl)[1,1biphenyl]-4-yl]methyl]-L-valine]; irbesartan [2-butyl-3-[[2′-(1H-tetrazol-5-yl) [1,1 ′-biphenyl]-4-yl]methyl]-1,3-dizaspiro [4,4] non-1-en-4-one]; candesartan [(±)-1-[[(cyclohexyloxy)carbonyl]oxy]ethyl-2-ethoxy-1-[[2′(1H-tetrazol-5-yl) [1,1′-biphenyl]-4-yl]methyl]-1H-benzimidazole-7-carboxylate]; telmisartan [4′-[(1,4′-dimethyls-2′-propyl-[2,6′-bi-1H-benzimidazol]-1′-yl)methyl]]-1,1′-biphenyl]-2-carboxylic acid]; eprosartan [E-α-[[2-butyl-1-[(4carboxyphenyl)methyl]-1H-imidazol-5 -yl]methylene]-2-thiophenepropanoic acid]; N-substituted imidazole-2-one (U.S. Pat. No. 5,087,634); imidazole acetate derivatives including 2-n-butyl-4-chloro-1-(2-chlorobenzyl) imidazole-5-acetic acid (see Wong et al., J. Pharmacol. Exp. Ther. 247(1):1-7 (1988)); 4, 5, 6, 7tetrahydro-1H-imidazo pyridine-6-carboxylic acid and derivatives (U.S. Pat. No. 4,816,463); N-2-tetrazole beta-glucorunide analogs (U.S. Pat. No. 5,085,992); substituted pyrroles, pyrazoles and triazoles (U.S. Pat. No. 5,081,127);phenyl and heterocyclic derivatives such as 1,3-imidazoles (U.S. Pat. No. 5,073,566); and imidazo-fused 7-member ring heterocycles (U.S. Pat. No. 5,064,825).

Additional angiotensin-II receptor antagonists include: peptides (e.g. U.S. Pat. No. 4,772,684); antibodies to angiotensin II (e.g. U.S. Pat. No. 4,302,386); aralkyl imidazole compounds such as biphenyl-methyl substituted imidazoles (i.e. EP No. 253,310, Jan. 20, 1988); ES-8891 (N-morpholinoacetyl-(-1-napthyl)-L-alanyl-(4-thiazolyl)-L-alanyl-(35, 45)-4-amino-3-hydroxy-5-cyclohexapentanoyl-n-hexylamide, Sankyo Company Ltd., Tokyo, Japan); SK&F 108566; remikirin (Hoffmann LaRoche AG), adenosine A₂ agonists (Marion Merrell Dow) and certain nonpeptide heterocycles (G.D. Searle & Company).

As used herein, “vasopressin receptor antagonist” is intended to refer to a compound that interferes with or competitively inhibits the binding of vasopressin to a vasopressin receptor. Preferred vasopressin receptor antagonists are VP-343 (Naito et al., Biol. Pharm. Bull. 23(2):182-89 (2000)) and OP-21268 (Nakamura et al., Eur. J. Pharmacol. 391(1-2):39-48 (2000)). The interaction of vasopressin receptor antagonists with vasopressin receptors has been described in detail by Tanaka et al. (Brain Res. 644(2):343-346 (1994)); Burrell et al. (Am. J. Physiol. 275:H176-H182 (1998)); and Chen et al. (Eur. J. Pharmacol. 376(1-2):45-51 (1999)).

The term “vasopressin receptor antagonist” is also intended to encompass the peptide vasopressin receptor antagonists as disclosed in Manning et al. (J. Med. Chem. 35:382-88 (1992)); Manning et al. (J. Med. Chem. 35:3895-904 (1992)); Gavras and Lammek (U.S. Pat. No. 5,070,187 (1991)); Manning and Sawyer (U.S. Pat. No. 5,055,448 (1991)); Ali (U.S. Pat. No. 4,766,108 (1988)); and Ruffolo et al. (Drug News and Perspective 4(4):217 (1991)). Williams et al. have also reported on potent hexapeptide oxytocin antagonists which also exhibit weak vasopressin antagonist activity in binding to vasopressin receptors (J. Med. Chem. 35:3905 (1992)).

The term “vasopressin receptor antagonist” is also intended to encompass the nonpeptide vasopressin receptor antagonists as disclosed in Yamamura et al. (Science 252:572-74 (1991)); Yamamura et al. (Br. J. Pharmacol. 105:787-791 (1992)), Ogawa et al. (Otsuka Pharm Co., LTD.); EP 0514667-Al; JP 04154765-A; EPO 382185-A2; and W09105549. Other nonpeptide vasopressin antagonists have been disclosed by Bock and Williams (EP 0533242A); Bock et al. (EP 0533244A); Erb et aL (EP0533240A); and K. Gilbert et al. (EP 0533243A).

As used herein, “angiotensin converting enzyme (ACE) inhibitor” is intended to refer to a compound that interferes with or inhibits the conversion of angiotensin I to angiotensin II in the renin-angiotensin system. Examples of ACE inhibitors include, but are not limited to, benzazepine compounds such as benazepril (3-[(1 -ethoxycarbonyl-3 -phenyl-(1S)-propyl]amino)-2,3,4,5 tetrahydro-2-oxo-1-1-(3S)-benzazepine-1-acetic acid, Ciba-Geigy Ltd., CGS 14824A), and libenzapril (3-[(5-amino-1-carboxy-1S-pentyl)amino],2,3,4,5tetrahydro-2-oxo-3S-1H-1-benzazepine-1-acetic acid, Ciba-Geigy Ltd., CGS 16617); 6H-pyridazino[1,2-a]diazepine derivatives such as cilazapril (Hoffmann-LaRoche, RO 31-2848); 2,3-dihydro-1H-indene compounds such as delapril (N[N-[(S)-1-ethoxycarbonyl-3-phenylpropyl]-L-alanyl]-N-(indan-2-yl)glycine, CV3317); L-proline derivatives such as alacepril (-[(S)-3-acetylthio-2methylpropanoyl]-L-prolyl-L-phenylalanine, DU-1219), altiopril (N-[3 -(N-cyclohexanecarbonyl-D-alanylthio)-2-methylpropanoyl]-L-proline, Chugai Pharmaceutical Co. Ltd., MC 838), captopril (D-3 -mercapto-2-methylpropanoyl-L-proline, Bristol-Myers Squibb, SQ 14,225), ceronapril ((S)-1-[6-amino2[[hydroxy(4-phenylbutyl)phosphinyl] oxy]-1-oxohexyl]-L-proline, BristolMyers Squibb, SQ 29,852), enalapril (N-[(S)-1-(ethoxycarbonyl)-3phenylpropyl]-L-Ala-L-Pro, MK 421), fosinopril (Bristol-Myers Squibb, SQ 28,555), lisinopril (MK 521), and spirapril (7-N-[1(S)-ethoxycarbonyl-3phenylpropyl]-(S)-alanyl-1,4-dithia-7-azaspiro[4,4]-nonane-8(S)-carboxylic acid, Schering-Plough Corporation, SCH 33844); oxoimidazoline derivatives such as imidapril ((-)-(4S)-3-[(2S)-2-[[(1S)-1-ethoxycarbonyl-3 -phenylpropyl]amino]propionyl]-1 -methyl-2-oxoimidazolidine-4-carboxylic acid, Tanabe Seiyaku Co. Ltd., TA-6366); iso-quinoline carboxylic acid derivatives such as moexipril (2-[2-(1 -ethoxycarbonyl)-3-phenylpropyl]amino-1 -oxopropyl]-6,7dimethoxy-1,2,3,4-tetrahydroisoquinoline-3-carboxylic acid(S,S,S), Syntex Research, RS-10085) and quinapril (3S-[2[R*(R*)]],3R*]-2-[2-[[1-(ethoxy carbonyl)-3-phenylpropyl]-amino]-1-oxopropyl]1,2,3,4-tetrahydro-3isoquinolinecarboxylic acid, CI-906); 1H-indole carboxylic acid derivatives such as pentopril (Ciba-Geigy Ltd., CGS 13945) and perindopril (S 9490-3); hexahydroindole carboxylic acid derivatives such as trandolapril (Centre de Recherches Roussel-Uclaf, RU 44570); cyclopenta[b]pyrrole carboxylic acid derivatives such as ramipril (2-[N-[(S)-1-ethoxycarbonyl-3-phenylpropyl]-Lalanyl]-(1S,3S,5S)-2-azabicyclo[3.3.0]octane-3-carboxylic acid, Hoechst Marion Roussel, Hoe 498); and 1,4-thiazepine compounds such as temocapril (alpha[(2S,6R)-6-[(1 S)-1 -ethoxycarbonyl-3-phenylpropyl]amino-5-oxo-2-(2thienyl)perhydro-1,4-thiazepin-4-yl]acetic acid, Sankyo Co. Ltd., CS-622).

As used herein, “blood-brain barrier” refers to the continuous wall formed by intercellular junctions between endothelial cell-comprising brain capillaries (Goldstein, et al., Scientific American 255:74-83 (1986); Pardridge, Endocrin. Rev. 7:314-330 (1986)) which prevent the passive movement of many molecules from the blood to the brain.

As used herein, “assessing” refers to the measuring of transient K⁺ currents in excitable cells by step voltage clamp protocols, described for example in Li and Ferguson, supra; Fedida and Giles, supra; and Bouchard and Fedida, supra.

As used herein, “isolated cell” is intended to refer to a cell that is substantially free from other cells with which the subject cell is typically found in its native state. The phrase “isolated cell” is also intended to refer to “isolated cell culture.”

II. Preferred Embodiments

The present invention is applicable to methods of treating patients who are suffering or who have suffered an ischemic event, and whose excitable cells are susceptible to damage as a result. Specific embodiments will be set forth in detail following a detailed explanation of the present invention.

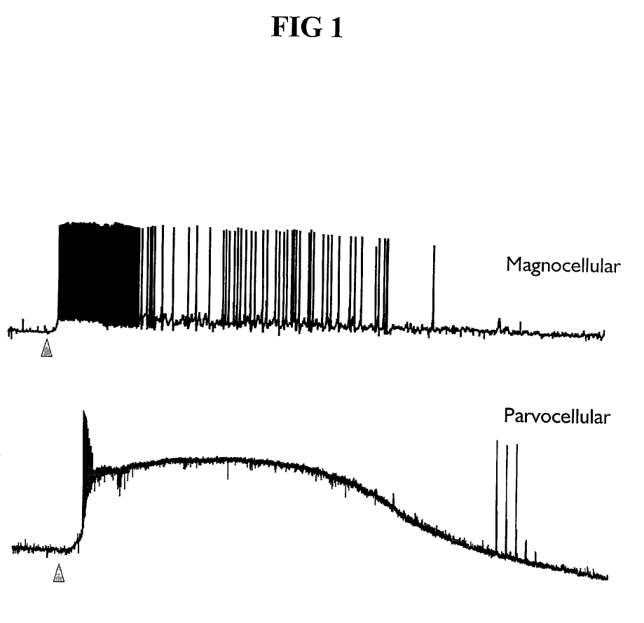

Excitotoxins that cause profound cell death in virtually all brain areas, including the parvocellular regions of the paraventricular nucleus of the hypothalamus (PVN) have been shown to have no effect on the viability of magnocellular neurosecretory cells of this nucleus (Herman and Wiegand, supra; Hastings and Herbert, supra). This selective cell death in vivo following microinjection of D-1-tetrazol-5yl-glycine, a specific NMDA agonist, correlates strongly with the electrophysiological response of the respective cell types to agonist application in an acute brain slice preparation (Bains and Ferguson, supra). The parvocellular neurons exhibit a rapid increase in firing frequency followed by a sustained depolarizing response following brief (1-2Seconds) application of NMDA agonist. This response, which has been classified previously as a long-duration plateau depolarization (LDPD), is similar to the extended neuronal depolarizations (END) described in hippocampal neurons and is a strong predictor of subsequent cell death (Sombati et al., Brain Res. 566:316319 (1991)). Conversely, magnocellular neurons, which are resistant to excitotoxic insult in vivo, do not exhibit such rapid sustained depolarizations (FIG. 1).

The instant inventors have shown that the dichotomy in responses between parvocellular and magnocellular neurons is not due to a difference in NMDA receptor kinetics resulting from variability in the heteromeric assembly of receptor subunits. Using voltage ramps, the instant inventors have discovered no appreciable difference, either in the degree of magnesium block, or in the amount of current passed at comparable membrane potentials, between the responses of magnocellular and parvocellular neurons.

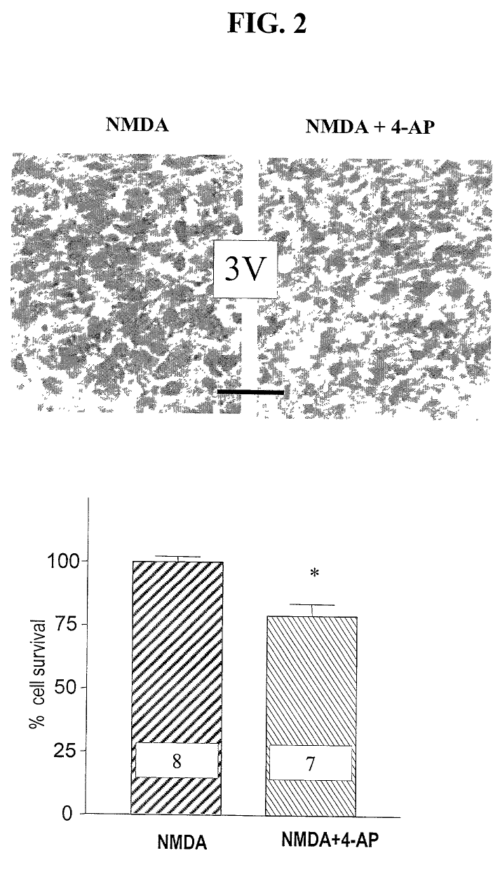

In the absence of any clear anatomical demarcation, differences in the intrinsic electrical properties of magnocellular and parvocellular neurons of PVN have been used as the primary tool to identify these cells during intracellular recordings. The former are characterized by the presence of a rapidly activatingrapidly inactivating K⁺ channel (Tasker and Dudek, supra; Li and Ferguson, supra). This current is important in membrane depolarization following action potentials and likely also regulates the interval between successive spikes (Connor and Stevens, J. Physiol. (London) 213:31-53 (1971)). It demonstrates similar pharmacological and biophysical properties to the delay current, I_{D}, which was so named because it delays the time to first spike (Storm, supra). This transient K⁺ current is also important in regulating neuronal excitability of magnocellular neurons in response to glutamate, and protects these cells from the overflow of glutamate that follows cerebral ischemia (Bains and Ferguson, supra). Inhibition of this conductance by 100 μM 4-AP dramatically alters the response of magnocellular neurons to NMDA agonist, from a small, depolarizing event to a prominent plateau potential in the presence of 4-AP, similar to that observed in parvocellular neurons which are not resistant to excitotoxicity (Id.). This effect of 4-AP is likely postsynaptic since an increase in neuronal excitability, as measured by spiking in response to depolarizing current pulses, is observed in 4-AP (Id.). Application of 4-AP also unmasks presumptive dendritic calcium spikes (Id.). In experiments evaluating cell death following microinjection of NMDA agonist with and without pretreatment by microinjection of 4-AP, a statistically significant reduction in magnocellular neuron numbers in PVN treated with 4-AP was observed (FIG. 2). Effectively the proportion of magnocellular neurons surviving (78.9±4.6 %) was now found to be equivalent to that observed in parvocellular neurons (80.9±3.0 %) (Id.). Meanwhile, 4-AP had no significant effect on the latter population of cells (Id.). Thus, the effects of 4-AP on magnocellular neuron cell excitability translate into definitive changes in the cells' ability to withstand excitotoxic challenge

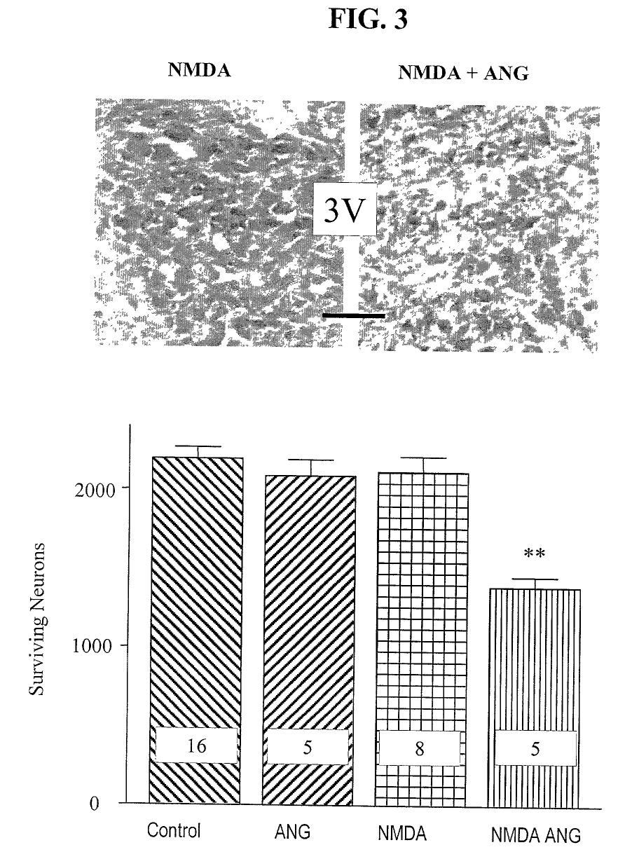

The discovery by the instant inventors of protection against excitotoxic cell death by a 4-AP-sensitive transient K⁺ conductance led to further experiments with alternative inhibitors of this conductance. Selective AT₁ receptor mediated inhibition of this transient K⁺ conductance of magnocellular neurons in PVN by angiotensin-II has been previously reported (Li and Ferguson, supra). It has been demonstrated that angiotensin-II administration in PVN slices has effects similar to 4-AP in increasing the number of action potentials in response to depolarizing current pulses (Bains and Ferguson, supra). Such actions would clearly result in an increased likelihood of the occurrence of LDPD. The functional consequence of such an effect is that microinjection of angiotensin-II into PVN prior to NMDA agonists eliminates the resistance to cell death normally observed in magnocellular neurons. Thus, only 80.8±3.75 % of magnocellular neurons survive following NMDA if preceded by angiotensin-II, while no cell death is observed following NMDA agonist (100.0±2.4 %) or angiotensin-II (97.4±4.2 %) alone (FIG. 3). These data demonstrate that this transient K⁺ conductance plays a dominant role in controlling the excitability of PVN magnocellular neurons, contributing to the resistance of these neurons to excitotoxic cell death.

One of the primary risk factors for stroke is hypertension, a clinical condition which is normally associated with increased circulating and central levels of angiotensin-II (Sambhi et al., Circ. Research 36 (6 Suppl. 1):28-37 (1975)). Increased levels of angiotensin-Il may exacerbate ischemia-induced cell death. Hypertensive treatments based on the blockade of angiotensin-II receptors have dramatic effects in prolonging life expectancy that cannot be explained simply by their blood pressure-lowering effects (Pit et al., Lancet 349:747-752 (1997)). The blockade of angiotensin-II receptors also decreases the frequency and severity of stroke in a variety of animal models at doses that have no effect on blood pressure (Stier et al., supra). As an alternative to blocking angiotensinII receptors, the hypertensive effects of angiotensin-II may be treated by preventing the conversion of angiotensin-I to angiotensin-II, carried out by ACE, in the renin-angiotensin pathway. This conversion may be prevented by administering an ACE inhibitor(s) to hypertensive subjects.

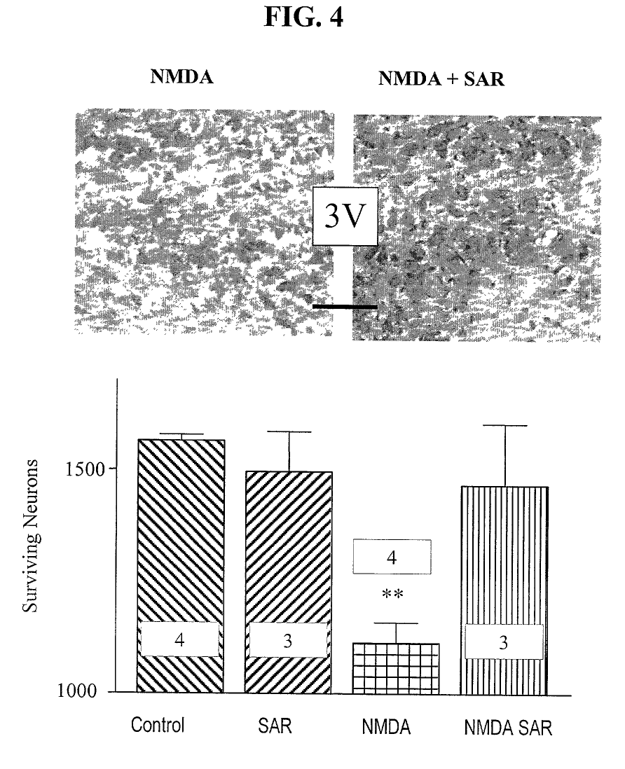

To determine whether magnocellular neurons in hypertensive rats with increased central angiotensin-II lose their resistance to excitotoxins as a consequence of endogenous angiotensin-II inhibiting the transient K⁺ conductance, NMDA agonist or vehicle control was microinjected into PVN and surviving neurons were counted three days later. Following such treatment, a similar loss of parvocellular neurons to that found in normotensive animals was observed (82±2% surviving), while the resistance of magnocellular neurons was no longer observed in these animals (71±5% surviving) (see FIG. 3 for specific N values). To confirm that angiotensin-II was responsible for this loss of resistance, the NMDA agonist was next microinjected into PVN of spontaneously hypertensive rats immediately following the angiotensin-II receptor antagonist saralasin. Under these conditions, magnocellular neurons were again found to be resistant to excitotoxic cell death with no observed cell loss three days later (96±10% surviving), while the parvocellular neurons were still significantly reduced in number (83±2% surviving) (see FIG. 4 for specific N values). These findings provide the first direct evidence that elevated angiotensin-II concentrations in the central nervous system of hypertensive animals may contribute to the increased susceptibility for stroke and that these actions can be prevented by central angiotensin-II receptor blockade.

The dominant role played by the transient K⁺ conductance in regulating the excitability of magnocellular PVN neurons provides resistance to glutamate-mediated excitotoxic cell death. This neuronal interaction between postsynaptic K⁺ conductances that regulate membrane excitability, and glutamate, represents a novel target for therapies directed toward reducing both the occurrence and consequences of stroke. Modulation of this conductance by 4-AP or angiotensin-II results in effects on the neurons' response to NMDA agonists in accordance with the invention. In contrast, enhancing the transient K⁺ conductance by inhibiting the actions of angiotensin-II may lower the probability and consequences of stroke. Pharmacological agents that inhibit AT₁ receptors consequently provide an unexpected benefit for patients afflicted with hypertension.

The present invention thus provides methods of treating patients who are suffering or who have suffered an ischemic event, and whose excitable cells are susceptible to damage as a result. More specifically, the present invention is applicable to preventing ischemia-induced cellular damage from occurring, arresting the development of ischemia-induced cellular damage, and relieving ischemia-induced cellular damage by administering a compound which increases a transient K⁺ current in the potentially affected cells. Although not meant to be limiting, among the cells potentially affected by ischemic events are the magnocellular neurons of the paraventricular nucleus of the hypothalamus. all other neurons, particularly those of the brain, cardiac myocytes, and all other excitable cells expressing a transient K⁺ conductance.

The present invention also provides in vivo and in vitro methods for screening for compounds that increase a transient K⁺ current in the excitable cells of a patient. The in vivo method for screening for such compounds comprises: (i) inducing ischemia in a subject; (ii) assessing a transient K⁺ current in the subject; (iii) administering to the subject an effective amount of a test compound; and (iv) assessing the transient K⁺ current in the subject. The in vitro method for screening for such compounds comprises: (i) inducing an oxygen-deprived state mimicking ischemia in an isolated cell; (ii) assessing a transient K⁺ current in the cell; (iii) administering to the cell an effective amount of a test compound; and (iv) assessing the transient K⁺ current in the cell. In both methods, an increase in the transient K⁺ current indicates that the test compound increases a transient K⁺ current in the excitable cells of a patient. Transient K⁺ currents in excitable cells may be assessed by step voltage clamp protocols as described in Li and Ferguson, supra; Fedida and Giles, supra; and Bouchard and Fedida, supra. Examples of appropriate subjects for inducing ischemia, both focal and global, and for screening for compounds which ameliorate ischemia-induced injury are provided in Inada et al., supra; Li et al., (Stroke 31(1):176-182 (2000)); and Takagi et al., (J. Cereb. Blood Flow Metab. 19(8):880-888 (1999)).

Compounds which are capable of increasing a transient K⁺ current include angiotensin-II receptor antagonists. Among these are: saralasin; losartan [2butyl-4-chloro-1-[p-(o-1H-tetrazol-5-ylphenyl)-benzyl]imidazole-5-methanol monopotassium salt]; valsartan [N-(1-oxopentyl)-N-[[2′-(1H-tetrazol-5-yl)[1,1′biphenyl]-4-yl]methyl]-L-valine]; irbesartan [2-butyl-3-[[2′-(1H-tetrazol-5-yl) [1,1′-biphenyl]-4-yl]methyl]-1,3-dizaspiro [4,4]non-1-en-4-one]; candesartan [(±)-1-[[(cyclohexyloxy)carbonyl]oxy]ethyl-2-ethoxy-1-[[2′(1 H-tetrazol-5-yl) [1,1′-biphenyl]-4-yl]methyl]-1H-benzimidazole-7-carboxylate]; telmisartan [4′[(1 ,4∝0-dimethyls-2′-propyl[2,6′-bi-1H-benzimidazol]-1 ′-yl)methyl]]-1,1′biphenyl]-2-carboxylic acid]; eprosartan [E-a-[[2-butyl-1-[(4carboxyphenyl)methyl]-1 H-imidazol-5 -yl]methylene]-2-thiophenepropanoic acid]; CGP 42112 A (Nasjletti and Mason, supra); N-substituted imidazole-2-one (U.S. Pat. No. 5,087,634); imidazole acetate derivatives including 2-n-butyl-4chloro-1-(2-chlorobenzyl) imidazole-5-acetic acid (see Wong et al., J. Pharmacol. Exp. Ther. 247(1):1-7(1988)); 4,5,6,7-tetrahydro-1H-imidazo [4,5c] pyridine-6-carboxylic acid and derivatives (U.S. Pat. No. 4,816,463); N-2tetrazole beta-glucorunide analogs (U.S. Pat. No.5,085,992); substituted pyrroles, pyrazoles and triazoles (U.S. Pat. No. 5,081,127); phenyl and heterocyclic derivatives such as 1,3-imidazoles (U.S. Pat. No.5,073,566); and imidazo-fused 7-member ring heterocycles (U.S. Pat. No. 5,064,825).

Additional angiotensin-II receptor antagonists include: peptides (e.g. U.S. Pat. No. 4,772,684); antibodies to angiotensin II (e.g. U.S. Pat. No. 4,302,386); aralkyl imidazole compounds such as biphenyl-methyl substituted imidazoles (e.g. EPNo.253,310, Jan. 20,1988); ES-8891 (N-morpholinoacetyl-(-1-napthyl)L-alanyl-(4-thiazolyl)-L-alanyl-(35, 45)-4-amino-3-hydroxy-5-cyclohexapentanoyl-n-hexylamide, Sankyo Company Ltd., Tokyo, Japan); SK&F 108566; remikirin (Hoffmann LaRoche AG), adenosine A₂ agonists (Marion Merrell Dow) and certain nonpeptide heterocycles (G.D. Searle & Company).

In a preferred embodiment of this invention, the angiotensin-II receptor antagonist is losartan. Losartan has been found to cross the blood-brain barrier (Li et al., Brain Res. Bull. 30:33-39 (1993)).

In another preferred embodiment of this invention, the angiotensin-II receptor antagonist is saralasin. Saralasin, unlike losartan, does not cross the blood-brain barrier (Li et al., supra).

Other compounds capable of increasing a transient K- current include vasopressin receptor antagonists.

The transient K⁺ currents that may be targeted by these compounds include the A current, the delay current, and I_{TO}. The modulating of transient K⁺ currents to treat disease has been disclosed in WO 98/16185. However, the invention disclosed in WO 98/16185 teaches away from the present invention in that it describes compounds which inhibit transient K⁺ currents.

In preventing damage to excitable cells during or following an ischemic event, compounds capable of increasing a transient K⁺ current can be co-administered with one or more agents active in reducing ischemia-induced damage or in preventing further ischemia from occurring, including thrombolytic agents such as recombinant tissue plasminogen activator (TPA) and streptokinase. Transient K⁺ current-increasing compounds such as angiotensin-II receptor antagonists may also be used in conjunction with agents which protect excitable cells from damage due to ischemia-induced energy deficit, such as glutamate antagonists and Ca²⁺ channel antagonists.

Transient K⁺ current-increasing compounds may also be administered in conjunction with antiplatelet agents such as aspirin, ticlopidine, or dipyridamole. These agents prevent ischemia by inhibiting the formation of intraarterial platelet aggregates that can form on diseased arteries, induce formation of clots, and occlude the artery. Compounds capable of increasing a transient K⁺ current may also be administered in conjunction with anticoagulant agents such as heparin, which are widely used in the treatment of transient ischemic attacks (Harrison 's Principles of Internal Medicine, 14th Ed., Vol. 2, p. 2337, McGraw-Hill (1998)).

Co-administration can be in the form of a single formulation (combining, for example, an angiotensin-II receptor antagonist and ticlopidine- with pharmaceutically acceptable excipients, optionally segregating the two active ingredients in different excipient mixtures designed to independently control their respective release rates and durations) or by independent administration of separate formulations containing the active agents.

Having now generally described the invention, the same will now be more readily understood by reference to the following examples, which are provided by way of illustration and are not intended to be limiting.

The disclosures of all patent documents and publications disclosed throughout the instant specification are hereby incorporated by reference in their entirety.

### EXAMPLES

### Example I

### Histology

Experiments were performed on male Sprague-Dawley rats (150-525 g. Charles River, P.Q., Canada). The animals were anesthetized with sodium pentobarbitol (65 mg/kg, ip), placed in a stereotaxic frame and the skull exposed, and a small burr hole drilled in the skull such that a cannula electrode (tip diameter 150 μm) could be advanced into the region of the PVN according to the coordinates of Paxinos and Watson (−0.9 mm Bregma, 0.5 mm lateral, 7.5 mm ventral) (Paxinos and Watson, The Rat Brain in Stereotaxic Coordinates, Academic Press, New York (1982)). Each animal received a 1.0 μl microinjection to each PVN (2×0.5 μl) according to one of the following protocols, saline/saline, saline/NMDA, 4-AP/saline, 4-AP/NMDA, angiotensin-II/saline, angiotensin-II/NMDA, saralasin (SAR)/NMDA. The incision was then closed and the animal received the analgesic Buprenorphin (0.03 mg/kg, SQ) to aid postoperative recovery. Animals were allowed to recover for three days after which they were overdosed with sodium pentobarbitol (100 mg/kg) and perfused with 0.9% saline followed by 10% formalin through the left ventricle of the heart. The brain was removed, placed in formalin overnight at 40° C. The brain was then cut into a smaller block contained PVN and stored in a 30% sucrose, 0.1 M phosphate buffer at 4° C. for at least two days.

The blocks were mounted, covered with Tissue-Tek O.C.T. compound (Sakura) and flash frozen in 2-methyl butane (cooled by dry ice) for 45 seconds. Using the Frigocut 280 (Reichart Jung), 20 μm coronal sections were cut through the area of PVN. These sections were mounted and cresyl violet stained. The histological locations of the microinjection sites were verified at the light microscope level by an observer unaware of the experimental conditions. Only those animals with microinjection within the boundaries of PVN were further analyzed.

Magnocellular neurons were differentiated from parvocellular neurons and cellular material by specific morphological characteristics (Sawchenko and Swanson, J. Comp. Neuro. 218:121-44 (1983)). In addition to the anatomical location of the neuron within PVN, morphological size was used to further characterize neuronal type. Neurons with soma diameter of approximately 20-25 μm and intact nuclei were characterized as viable magnocellular neurons. Neurons with soma diameter of between 15 and 20 μm were not included in the study, as they could not be reliably classified as belonging to either subpopulation. Histological sections were viewed under high magnification (40×) at the light microscope level and a grid was superimposed over each area of PVN. This superimposed grid was used to respectively count magnocellular and parvocellular neurons. In order to prevent the double counting of neurons, a neuron that came to lie on a vertical grid-line was deemed to belong to the grid to the immediate right, and a neuron that came to lie on a horizontal grid-line was deemed to belong to the grid directly above it. Following this method, a sum of the sections was established for magnocellular and parvocellular neurons from each hemisphere of PVN. Comparative analyses were performed whereby neurons were counted in 20 μm sections following the initial emergence of PVN. All counts given in FIGS. 2, 3 and 4 incorporate Abercrombie's correction for double counting (Coggeshall, Trends Neurosci. 15:9-13 (1992)).

### Example 2

### Electrophysiology

Male, Sprague-Dawley rats (150-250 g, Charles River, P.Q., Canada) were killed by decapitation, the brain was removed quickly from the skull and immersed in cold (1-4° C.) artificial cerebrospinal fluid (aCSF). The brain was blocked and 400 μm hypothalamic slices, which included the PVN, were prepared as described in Bains and Ferguson (NeuroReport 8(9-10):2101-05 (1997)). Slices were incubated in oxygenated aCSF (95% O₂, 5% CO₂) for at least 90 minutes at room temperature. Twenty minutes prior to recording, the slice was transferred into a modified interface type recording chamber and continuously perfused with aCSF at a rate of 1 ml/min.

Whole cell recordings were obtained using pipettes (resistance of 4-6 MQ) filled with a solution containing (in mM): Kgluconate (140), CaCl₂ (0.1), MgCl₂ (2), EGTA (1.1), HEPES (N-2-hydroxyethylpiperazine-N′-2ethanesulfonic acid) (10), Na₂ATP (2), and adjusted to pH 7.25 with KOH. The aCSF composition was (in mM): NaCl (124), KCl (2), KPO₄ (1.25), CaCl₂ (2.0), MgSO₄ (1.3), NaHCO₃ (20), and glucose (10). Osmolarity was maintained between 285 and 300 mOsm and pH between 7.3 and 7.4. A Ag-AgCl electrode connected to the bath solution via a KCl-agar bridge served as reference. All signals were processed with an Axoclamp-2A amplifier. For voltage clamp recordings, the continuous single-electrode voltage clamp configuration was used. Outputs from the amplifier were digitized using the C.E.D. 1401 plus interface and stored on computer for off-line analysis.

For isolated neurons, pipettes of 1-4 MΩ were filled with a pipette solution containing (in mM): potassium-gluconate (130), EGTA(10), MgCl₂ (1), HEPES (10), Na₂ATP (4), GTP (0.1), adjusted to pH 7.2 with KOH. The standard bath solution contained (inmM): NaCl (140), KCl (5), MgCl₂ (1), CaCl₂ (2), HEPES (10), glucose (10) and 11 M tetrodotoxin (TTX) (Alamone Labs, Jerusalem, Israel). Signals were amplified, collected and processed using an Axopatch 200B (Axon Instruments) amplifier, a 1401plus A-D interface and Signal software from C.E.D.

### Example 3

### The Presence of I_{D} Current in PVN Neurons

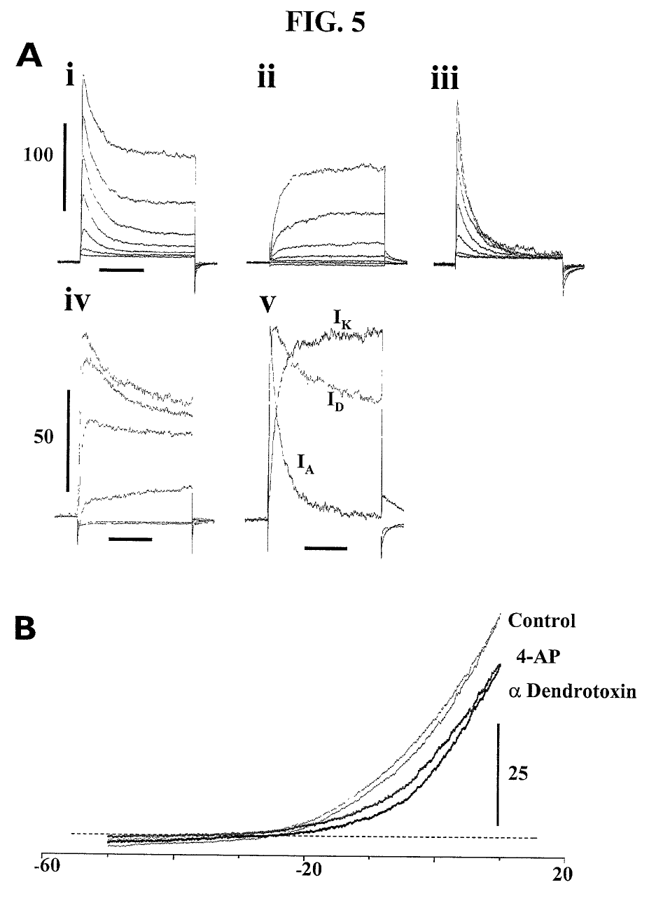

We obtained voltage clamp recordings from dissociated PVN neurons and observed the presence of a rapidly activating, slowly inactivating current that is distinct from I_{A} and is also sensitive to micromolar doses of 4-AP (Storm, Nature 336:379-81 (1988)) and submicromolar concentrations of a-dendrotoxin (a-DTX) I_{D}. A standard IV protocol (250 ms pulses between −100 and 10 mV), from a holding potential of −100 mV activates a family of outwardly-rectifying K⁺ currents exhibiting rapid activation and inactivation kinetics (a(i)) in isolated PVN neurons. Increasing the holding potential (−60 mV) leads to activation of K⁺ currents that exhibit slower activation kinetics and no inactivation (a(ii)) (I_{K}) The rapidly activating and inactivating component was obtained by arithmetic subtraction of a(i)−a(ii) and represents the I_{A} shown in a(iii). The family of K⁺ currents obtained by subtracting a family of currents similar to a(i) in the presence of 100 μM 4-AP from non-blocked currents represents the I_{D} current (a(iv)). Normalized traces at the same potential (10 mV) emphasize the difference in the activation and inactivation characteristics of the 3 K⁺ currents (a(v)) (FIG. 5a). Voltage ramps (100 mV/sec) activate an outwardly rectifying whole-cell current. This current is inhibited by 100 μM 4-AP, and to an equal degree by 1 μM a-DTX. The remaining current is I_{D} (FIG. 5b).

What is claimed is:

1. A method of preventing damage to the excitable cells of a patient which comprises administering to said patient during or after said patient undergoes or has undergone an ischemic event, an effective amount of a compound which increases a transient potassium (K⁺ ) current in the excitable cells of said patient.

2. The method of preventing damage to the excitable cells of a patient as claimed in claim 1, wherein said excitable cells are the neurons of the brain.

3. The method of preventing damage to the excitable cells of a patient as claimed in claim 1, wherein said excitable cells are the magnocellular neurons of the paraventricular nucleus of the hypothalamus.

4. The method of preventing damage to the excitable cells of a patient as claimed in claim 1, wherein said transient K⁺ current is I_{A}

5. The method of preventing damage to the excitable cells of a patient as claimed in claim 1, wherein said transient K⁺ current is I_{D}.

6. The method of preventing damage to the excitable cells of a patient as claimed in claim 1, wherein said transient K⁺ current is I_{A} and I_{D}.

7. The method of preventing damage to the excitable cells of a patient as claimed in claim 1, wherein said K⁺ current is I_{TO}.

8. The method of preventing damage to the excitable cells of a patient as claimed in claim 1, wherein said compound crosses the blood-brain barrier.

9. The method of preventing damage to the excitable cells of a patient as claimed in claim 1, wherein said compound is a vasopressin receptor antagonist.

10. The method of preventing damage to the excitable cells of a patient as claimed in claim 9, wherein said vasopressin receptor antagonist crosses the blood-brain barrier.

11. The method of preventing damage to the excitable cells of a patient as claimed in claim 1, wherein said compound is an angiotensin converting enzyme (ACE) inhibitor.

12. The method of preventing damage to the excitable cells of a patient as claimed in claim 1 1, wherein said angiotensin converting enzyme (ACE) inhibitor crosses the blood-brain barrier.

13. The method of preventing damage to the excitable cells of a patient as claimed in claim 1, wherein said compound is angiotensin-II receptor antagonist.

14. The method of preventing damage to the excitable cells of a patient as claimed in claim 13, wherein said excitable cells are the magno cellular neurons of the paraventricular nucleus of the hypothalamus.

15. The method of preventing damage to the excitable cells of a patient as claimed in claim 13, wherein said transient K⁺ current is I_{A}.

16. The method of preventing damage to the excitable cells of a patient as claimed in claim 13, wherein said transient K⁺ current is I_{D}.

17. The method of preventing damage to the excitable cells of a patient as claimed in claim 13, wherein said transient K⁺ current is I_{A} and I_{D}.

18. The method of preventing damage to the excitable cells of a patient as claimed in claim 13, wherein said angiotensin-II receptor antagonist crosses the blood-brain barrier.

19. The method of preventing damage to the excitable cells of a patient as claimed in claim 13, wherein said angiotensin-II receptor antagonist is losartan.

20. The method of preventing damage to the excitable cells of a patient as claimed in claim 13, wherein said angiotensin-II receptor antagonist is saralasin.

21. An in vivo method for screening for a compound that increases a transient potassium (K⁺) current in the excitable cells of a patient, comprising the steps of:
(a) inducing ischemia in a subject;
(b) assessing a transient K⁺ current in said subject;
(c) administering to said subject an effective amount of a test compound; and
(d) assessing said transient K⁺ current in said subject, wherein an increase in said transient K⁺ current indicates that said test compound increases a transient K⁺ current in the excitable cells of a patient.

22. An in vitro method for screening for a compound that increases a transient K⁺ current in the excitable cells of a patient, comprising the steps of:
(a) inducing an oxygen-deprived state mimicking ischemia in an isolated cell;
(b) assessing a transient K⁺ current in said cell;
(c) administering to said cell an effective amount of a test compound; and
(d) assessing said transient K⁺ current in said cell, wherein an increase in said transient K⁺ current indicates that said test compound increases a transient K⁺ current in the excitable cells of a patient.
